(19) United States
(12) Patent Application Publication
(10) Pub. No.: US 2016/0287644 A1
(43) Pub. Date: Oct. 6, 2016

(54) FORMULATIONS FOR BENEFICIALLY ALTERING THE MICROBIOME

(71) Applicant: Vitamedica Corporation, Manhattan Beach, CA (US)

(72) Inventor: David H. Rahm, Manhattan Beach, CA (US)

(21) Appl. No.: 15/087,426

(22) Filed: Mar. 31, 2016

Related U.S. Application Data
(60) Provisional application No. 62/142,344, filed on Apr. 2, 2015.

(51) Int. Cl. A61K 35/745; A61K 35/747

(52) U.S. Cl. CPC A61K 35/745; A61K 35/747; A23L 1/3002; A23L 1/3014; A23L 1/3056; A23L 1/304; A23L 1/302; A23L 1/3006; A23L 1/3081; A23L 1/3082; A23V 2002/00; A23Y 2220/67; A23Y 2220/73; A23Y 2220/37; A23Y 2300/45; A23Y 2300/49; A23Y 2300/29

(57) ABSTRACT

Several embodiments of the invention provide a unique system of formulations adapted to beneficially alter the microbiome. In one embodiment, the invention comprises four oral formulations designed to work synergistically to assist in weight management. The formulations include three or four of the following: a cleansing formulation, a prebiotic formulation a probiotic formulation and a nutritional formulation. The formulations can be designed to work together to fundamentally change the flora of the gastrointestinal tract to achieve significant benefits.

### RELATED CASES

This application claims the benefit of U.S. Provisional Application No: 62/142,344, filed Apr. 2, 2015, the entirety of which is incorporated by reference herein.

### BACKGROUND

Dietary supplements have long been used to promote health and wellness. However, a need still exists for a formulation or a combination of formulations to effectively restore or rebalance the microbiome in a manner designed to cleanse, nourish, replenish, feed and boost the microbiome to facilitate weight management and/or promote other health benefits.

### SUMMARY

According to several embodiments, the invention comprises four oral formulations designed to work synergistically to beneficially alter the microbiome. In several embodiments, the invention comprises a set of formulations to uniquely assist in weight management. In addition to effective weight management, as a result of beneficially balancing the intestinal flora, associated health benefits are also provided.

In several embodiments of the invention, the system (or kit) of formulations comprises the following: a cleansing formulation, a probiotic formulation and a nutritional formulation. A prebiotic formulation may also be included. In several embodiments of the invention, the system (or kit) of formulations comprises the following: a cleansing formulation, a prebiotic formulation and a probiotic formulation.

In several embodiments, the first formulation is a cleansing formulation that is designed to provide a foundational “clean slate” to favorably alter the microbiome. The cleansing formulation, according to several embodiments, includes a (i) natural herbal laxative which helps restore and promote regularity (e.g., cascara sagrada), (ii) antibacterial herbs (such as yellow dock, licorice root, oregano leaf, and Oregon grape) to provide balance and cleansing support; (iii) milk thistle or similar ingredient to help eliminate toxins and excess waste and to protect and promote liver health, which in turn assists the detoxification process, (iv) carminative herbs (e.g., ginger and peppermint) to help soothe and calm the digestive system and minimize gastrointestinal discomfort, (v) a high mucilage ingredient (e.g., marshmallow) to coat, lubricate and sooth the digestive tract, and (vi) bitter herbs (e.g., burdock and dandelion) to stimulate digestive function and promote growth of beneficial bacteria through their inulin content.

In several embodiments, the second formulation is a prebiotic formulation that is designed to promote the growth and function of beneficial bacteria. The prebiotic formulation is optionally provided in some embodiments. The prebiotic formulation, according to several embodiments, includes (i) soluble fibers that stimulate the growth of beneficial species in the digestive tract, slow the rate of digestion, increase satiety, regulate blood sugar levels and beneficially affect total cholesterol, LDL cholesterol, triglycerides (e.g., glucomannan, a prebiotic fiber derived from Konjac root and inulin, a prebiotic fiber derived from Jerusalem artichoke); and (ii) insoluble fibers to add bulk to stool and promote regularity (e.g., rice bran powder and barley grass powder). In several embodiments, resistant maltodextrin, such as that sold under the trademark Fibersol-2® (Matsutani Chemical Industry Co., Ltd) or alternate, is included in addition to or as a substitute for several or all of the ingredients in the prebiotic formulation. In one embodiment, the prebiotic comprises or consists essentially of resistant maltodextrin.

In several embodiments, the third formulation is a probiotic formulation that is designed to rebalance the microbiome with beneficial bacteria that, for example, support a healthy weight. The probiotic formulation, according to several embodiments, includes (i) two or three species of Lactobacillus (e.g., selected from L. plantarum, L. rhamnosus, and L. gasseri) to support a healthy weight; (ii) two or more species of Bifidobacterium (e.g., selected from B. infantis, B. lactis, and B. breve) to increase the bacterial diversity in the GI tract; and (iii) inulin to provide optimum synbiotic support. In one embodiment, L. gasseri and L. rhamnosus are included to decrease abdominal visceral and subcutaneous fat areas or assist in overall weight loss. In several embodiments, the probiotics are in the range of 10-30 billion CFUs and are formulated (e.g., coated, encapsulated, etc.) to ensure that the probiotic organisms reach the intestines alive by shielding them from stomach acid. In one embodiment, the probiotic formulation is time-released to deliver at least 15 billion live bacteria throughout the digestive tract.

In several embodiments, the fourth formulation is a nutritional formulation that is designed to maintain the beneficial bacterial in the GI tract. The nutritional formulation can serve as a meal replacement and, according to several embodiments, includes (i) a soluble fiber (such as Fibersol-2® or alternate) in the form of a resistant starch that supports a healthy weight by creating a sense of fullness, delaying post-meal hunger, and slowing the rise of blood sugar after a meal; (ii) a high-quality protein (e.g., whey protein concentrate) to support lean muscle mass, and (iii) one or more vitamins and/or minerals. The fourth formulation can be provided as a powder, gel or liquid form. In powder form, the fourth formulation can be designed to mix easily with water, soy milk, almond milk, cashew milk, coconut water or milk, dairy milk and other liquids.

In one embodiment of the invention, a three or four-step program is provided in a kit with the following:

Step 1: a cleansing formulation to support the body's natural gastrointestinal detoxification processes, enhance digestive health and promote regularity.

Step 2 (may be excluded in a three-step program): a prebiotic formulation to nourish the gut microflora and promote a better balance of beneficial bacteria.

Step 3: a probiotic formulation to boost the number and diversity of beneficial bacteria in the digestive tract by providing 10-25 billion CFUs (e.g., 16 billion CFUs) and nourish them.

Step 4: a nutritional formulation that serves as a nutritionally-balanced meal replacement shake to promote satiety and nutrient intake.

In several embodiments of the invention, the system (or kit) of formulations comprises the following: a cleansing formulation, a prebiotic formulation and a probiotic formulation, each as described herein.

The formulations described herein can be beneficial to both maintain a healthy microbiome and correct a damaged microbiome. In several embodiments, use of the formulations described herein provides one or more of the following advantages: (i) weight loss, (ii) maintenance of a healthy weight, (iii) regularity, (iv) antimicrobial effects with respect to undesired microorganisms, (v) anti-inflammatory, (vi) promotion of beneficial bacterial growth and function, (vii) satiety, (viii) regulation of glucose levels, (ix) reduced LDL and triglycerides, (x) increased muscle tone, and (xi) increased thermogenesis. Other advantages may also be present in addition to (or in place of) any of the above. For example, in one embodiment, the use of the formulations described herein reduces levels of C-reactive protein.

In several embodiments, use of the cleansing, prebiotic and probiotic formulations (and optionally the nutritional formulation) described herein increase the number of beneficial bacteria by 10-100% (e.g., 10%, 25%, 50%, 75%, 100%, 250% and ranges in between) after use. In some cases, a 3-20 fold increase in beneficial bacteria is achieved after using some embodiments of the invention. Undesired strains of micro-organisms (including bacteria, yeast, etc.) are reduced by 10-100% (e.g., 10%, 25%, 50%, 75%, 100%, 250% and ranges in between) after use of the cleansing, prebiotic and probiotic formulations (and optionally the nutritional formulation) described herein.

Various portions of the ingredients described herein may be used (e.g., root, seed, bark, leaf, flower, extracts, oils, etc.). As an example, if a root is provided in some embodiments, another portion of the plant may be substituted in other embodiments. Thickeners, binders, fillers, stabilizers and other excipients may be included in the formations described herein. Compounds to balance pH may also be included. Flavorings or preservatives may be used. In one embodiment, the formulations described herein can be manufactured with reduced or no synthetic preservatives. In some embodiments, the formulations are free of artificial colors and/or artificial flavors. In some embodiments, the formulations are free of known or common allergens. Soy-free, gluten-free, lactose-free and/or vegetarian formulations are provided in some embodiments. Some or all ingredients may be organic and/or non-GMO. In one embodiment, one or more of the formulations are vegan.

The use of the term ingredients and compounds may be used interchangeably herein. The terms composition and formulation can be used interchangeably. Where percentages are provided ingredients and compounds, they can be % m/m, % m/v or % v/v with respect to the specific formulation as a whole, unless otherwise indicated,

The agents, ingredients and compounds described herein may be modified natural substances (e.g., isolates, extracts, purified, processed, chemically modified, etc.) or synthetic substances. Methods of using unique combinations of all-natural substances are also provided.

In several embodiments, the invention comprises a method of beneficially affecting the microbiome using any one of the formulations described above or below. The use of any of the formulations described herein for treating assisting in weight management is provided herein. Individuals with a healthy BMI may benefit from the formulations described herein. For example, enhanced immunity and other health benefits may be provided as a direct or indirect result of a healthy microbiome.

### DETAILED DESCRIPTION

As briefly described above, in several embodiments, the invention comprises several formulations that work together in a synergistic manner to affect the microbiome, which in turn, can facilitate weight management and provide additional benefits,

In several embodiments of the invention, the formulations of the inventions comprise the following formulations: a cleansing formulation, a prebiotic formulation, a probiotic formulation and a nutritional formulation,

In one embodiment, the first formulation is a cleansing formulation and comprises or consists essentially of: cascara sagrada (bark), Oregon grape (root), milk thistle extract (80% silymarin) (seed), oregano leaf, peppermint leaf, licorice root, ginger (root), marshmallow (root), burdock (root), dandelion (root), and yellow dock (root). Vegetable cellulose may also be included depending on the embodiment.

In one embodiment, the second formulation is a prebiotic formulation and comprises or consists essentially of: glucomannan (from konjac root), rice bran powder, barley grass powder, and Jerusalem artichoke (extract) (90% inulin). Gelatin, magnesium stearate, microcrystalline cellulose and silicon dioxide may also be included. In several embodiments, resistant maltodextrin (such as Fibersol-2® or alternate) is included in addition to or as a substitute for several or all of the ingredients in the prebiotic formulation. In one embodiment, the prebiotic consists or consists essentially of resistant maltodextrin. In some embodiments, the prebiotic is not included in a system of formulations that includes the cleansing formulation the probiotic and the nutritional formulations. In several embodiments, the system (or kit) of formulations includes the cleansing formulation, the prebiotic formulation and the probiotic formulation.

In one embodiment, the third formulation is a probiotic formulation and comprises or consists essentially of two or more, or all of the following: Lactobacillus plantarum, Lactobacillus rhamnosus, Lactobacillus gasseri, Bifidobacterium infantis, Bifidobacterium lactis, and inulin. In another embodiment, the third formulation is a probiotic formulation and comprises or consists essentially of: Lactobacillus plantarum, Lactobacillus rhamnosus, Lactobacillus gasseri, Bifidobacterium infantis, Bifidobacterium lactis, Bifidobacterium breve, and inulin. Microcrystalline cellulose, hydroxypropyl methylcellulose, pectin, sodium carbonate, anhydrous, stearic acid, guar gum, and turmeric (color) may also be included.

In one embodiment, the fourth formulation is a nutritional formulation and comprises or consists essentially of: whey protein concentrate, resistant maltodextrin (such as Fibersol-2® or alternate), erythritol, sunflower oil, natural and artificial flavors, vitamin and mineral blend (calcium phosphate, magnesium oxide, ferrous fumarate, ascorbic acid, Dl-alpha tocopheryl acetate, molybdenum amino acid chelate, selenium amino acid chelate, niacinamide, zinc oxide, retinyl acetate, copper gluconate, calcium pantothenate, manganese sulphate, cholecalciferol, pyridoxine hydrochloride, thiamin hydrochloride, chromium polynicotinate, cyanocobalamin, potassium iodide), cellulose gum, xanthan gum, carrageenan, salt, maltodextrin, stevia and monk fruit extract. Cocoa may also be included depending on the embodiment.

In several embodiments, (i) the first formulation is a cleansing formulation and comprises or consists essentially of cascara sagrada, Oregon grape, milk thistle extract, oregano leaf, peppermint leaf, licorice root, ginger, marshmallow, burdock, dandelion, and yellow dock; (ii) the second formulation is a prebiotic formulation and comprises or consists essentially of glucomannan, rice bran powder, barley grass powder, and Jerusalem artichoke the third formulation is a probiotic formulation and comprises or consists essentially of Lactobacillus plantarum, Lactobacillus rhamnosus, Lactobacillus gasseri, Bifidobacterium infantis, Bifidobacterium lactis, and optionally Bifidobacterium breve, and inulin; and (iv) the fourth formulation is a nutritional formulation and comprises or consists essentially of: whey protein concentrate, resistant maltodextrin, erythritol, sunflower oil, vitamin and mineral blend (e.g., calcium phosphate, magnesium oxide, ferrous fumarate, ascorbic acid, Dl-alpha tocopheryl acetate, molybdenum amino acid chelate, selenium amino acid chelate, niacinamide, zinc oxide, retinyl acetate, copper gluconate, calcium pantothenate, manganese sulphate, cholecalciferol, pyridoxine hydrochloride, thiamin hydrochloride, chromium polynicotinate, cyanocobalamin, and potassium iodide), a sweetener, and optionally cocoa. In several embodiments, resistant maltodextrin (such as Fibersol-2® or alternate) is included in addition to or as a substitute for several or all of the ingredients in the prebiotic formulation. In one embodiment, the prebiotic consists or consists essentially of resistant maltodextrin.

In one embodiment, a system of three or four formulations comprises or consists essentially of the following:
(i) A cleansing formulation including cascara sagrada, Oregon grape, milk thistle extract, oregano leaf, peppermint leaf, licorice root, ginger, marshmallow, burdock, dandelion, and yellow dock.
(ii) A prebiotic formulation including glucomannan, rice bran powder, barley grass powder, and Jerusalem artichoke. In several embodiments, resistant maltodextrin (such as Fibersol-2® or alternate) is included in addition to or as a substitute for several or all of the ingredients in the prebiotic formulation. In one embodiment, the prebiotic comprises or consists essentially of resistant maltodextrin. The prebiotic formulation is excluded in some embodiments of a three-step system.
(iii) A probiotic formulation including Lactobacillus plantarum, Lactobacillus rhamnosus, Lactobacillus gasseri, Bifidobacterium infantis, Bifidobacterium lactis, optionally Bifidobacterium breve, and optionally inulin.
(iv) A nutritional formulation including whey protein concentrate, resistant maltodextrin, erythritol, a plant-based oil, and a vitamin and mineral blend. The nutritional formulation is excluded in some embodiments of a three-step system.

In one embodiment, particular ranges of the ingredients are provided. Non-limiting examples of these ranges are provided below.

In one embodiment, the cleansing formulation comprises or consists essentially of:

Amount Per Serving (A serving may be, for example, 2 capsules)

Cascara Sagrada (bark) 100-1000 mg
Cleansing Herbal Blend
Oregon Grape (root) 10-500 mg
Milk Thistle Extract (80% Silymarin) (seed) 10-500 mg
Oregano Leaf 10-500 mg
Peppermint Leaf 10-500 mg
Licorice Root 10-500 mg
Ginger (root) 5-300 mg
Digestive Blend
Marshmallow (root) 10-500 mg
Burdock (root) 10-500 mg
Dandelion (root) 10-500 mg
Yellow Dock (root) 10-500 mg

In one embodiment, the prebiotic formulation comprises or consists essentially of:

Amount Per Serving (A serving may be, for example, 2 capsules)

Glucomannan (from Konjac Root) 750-2,000 mg
Rice Bran Powder 50-100 mg
Barley Grass Powder 50-100 mg
Jerusalem Artichoke (extract) (90% inulin) 4-20 mg
In several embodiments, resistant maltodextrin
(such as Fibersol-2 ® or alternate) is included in addition to or as a
substitute for several or all of the ingredients in the prebiotic formulation.
In one embodiment, the prebiotic consists or consists
essentially of resistant maltodextrin in a range of 750-2,000 mg.

In one embodiment, the probiotic formulation comprises or consists essentially of:

Amount Per Serving may be, for example, 5-25 billion CFU per caplet

Lactobacillus plantarum 20-50%
Lactobacillus rhamnosus 10-40%
Lactobacillus gasseri 5-25%
Bifidobacterium infantis 2-25%
Bifidobacterium lactis 2-25%
Optionally Bifidobacterium breve 2-25%
Inulin 10-500 mg

In one embodiment, the fourth formulation is a nutritional formulation and comprises or consists essentially of:

Amount Per Serving may be, for example, 43 g/8 oz water

Fat 0.5-10 g
Sodium 25-300 mg
Potassium 25-300 mg
Total Carbohydrates 3-25
Dietary Fiber 1-15 g
Protein 5-40 g
Optionally, a combination of some or all of the following is included:
Vitamin A (as retinyl acetate)
Vitamin C (as ascorbic acid)
Calcium (as calcium phosphate)
Iron (as ferrous fumarate)
Vitamin D (as cholecalciferol)
Vitamin E (as dl-alpha tocopheryl acetate)
Thiamin (as thiamin hydrochloride)
Riboflavin
Niacin (as niacinamide)
Vitamin B6 (as pyridoxine hydrochloride)
Folate
Vitamin B12 (as cyanocobalamin)
Biotin
Pantothenic Acid (as calcium pantothenate)
Phosphorous (as calcium phosphate)
Iodine (as potassium iodide)
Magnesium (as magnesium oxide)
Zinc (as zinc oxide)
Selenium (as selenium amino acid chelate)
Copper (as copper gluconate)
Manganese (as manganese sulphate)
Chromium
Molybdenum (as molybdenum amino acid chelate)

In one embodiment, a system of three or four formulations comprises or consists essentially of the following:

(i) A cleansing formulation including cascara sagrada (100-400 mg, e.g., 150, 250, 350 mg), Oregon grape (100-400 mg, e.g., 110, 130, 150, 200 mg), milk thistle extract (50-150 mg, e.g., 60, 80, 120 mg), oregano leaf (50-150 mg, e.g., 60, 80, 120 mg), peppermint leaf (20-100 mg, e.g., 50, 75, 100 mg), licorice root (20-100 mg, e.g., 50, 75, 100 mg), ginger (5-75 mg e.g., 10, 20, 30 mg), marshmallow (50-200 mg, e.g., 50, 100, 150 mg), burdock (50-150mg, e.g., 60, 80, 120 mg), dandelion (50-150 e.g., 150, 250, 350 mg, e.g., 60, 80, 120 mg) and yellow dock (50-150 mg, e,g., 60, 80, 120 mg), with ranges in between provided for each ingredient in some embodiments.

(ii) A prebiotic formulation in the range of about 400-3000 mg per serving, including glucomannan, rice bran powder, barley grass powder, and Jerusalem artichoke. Each of these ingredients, in some embodiments, is provided in the range of 100-1000 mg (e.g., 100-250, 250-500, 500, 500-750, 750-1000 mg) each, with ranges in between provided for each ingredient in some embodiments. In several embodiments, resistant maltodextrin (such as Fibersol-2® or alternate) is included in addition to or as a substitute for several or all of the ingredients in the prebiotic formulation. In one embodiment, the prebiotic consists or consists essentially of resistant maltodextrin. The prebiotic formulation is excluded in some embodiments of a three-step system.

(iii) A probiotic formulation including Lactobacillus plantarum (20-60%, e.g., 20, 40, 50%), Lactobacillus rhamnosus (10-40%, e.g., 15, 25, 35%), Lactobacillus gasseri (5-30%, e.g., 10, 15, 25%), Bifidobacterium infantis (5-20%, e.g., 5, 10, 15%), Bifidobacterium lactis (5-20%, e.g., 5, 10, 15%), optionally Bifidobacterium breve (5-20%, e.g., 5, 10, 15%), and inulin (25-250 mg, e.g., 50, 100, 150 mg). The percent ranges are provided with respect to the total bacterial content. In some embodiments, a probiotic formulation comprises two or more of the following: 2-20 billion Lactobacillus plantarum, 2-15 billion Lactobacillus rhamnosus, 0.5-10 billion Lactobacillus gasseri, 0.5-5 billion Bifidobacterium infantis, 0.5-5 billion Bifidobacterium breve, and 0.5-5 billion Bifidobacterium lactis, with ranges in between provided for each ingredient in some embodiments.

(iv) A nutritional formulation including: whey protein concentrate (10-30 g, e.g., 15, 18, 20 g), resistant maltodextrin, erythritol, a plant-based oil, a vitamin and mineral blend, and optionally cocoa. The fiber content may be in the range of 4-12 g (e.g., 5, 8, 10 g). The fat content may be in the form of healthy oils and in the range of about 1-5 g (e.g., 1, 2, 5 g). Total carbohydrate may be in the range of about 5-15 g (e.g., 9, 11, 14 g), with ranges in between provided for each ingredient in some embodiments. The vitamins and minerals provided in some embodiments are provided in a range of 5-30% (e.g., 10-25%) of the daily recommended intake. In some embodiments, a system of three formulations is provided, comprising:

(i) A cleansing formulation including, per serving size (e.g., two capsules or like), 100-400 (e.g., 250) mg cascara sagrada, 200-600 (e.g., 410) mg of an herbal blend (comprising or consisting essentially of Oregon grape root, milk thistle extract, oregano leaf, peppermint leaf, licorice root and ginger root), and 100-500 mg (e.g., 340) mg of a digestive blend (comprising or consisting essentially of marshmallow root, burdock root, dandelion root, and yellow dock root, with ranges in between provided for each ingredient in some embodiments.

(ii) A probiotic formulation including, per serving size (e.g., two caplets, spheres, or like), Lactobacillus plantarum, Lactobacillus rhamnosus, Lactobacillus gasseri (5-Bifidobacterium infantis, Bifidobacterium lactis and optionally Bifidobacterium breve and 50-200 (e.g., 100) mg inulin.

(iii) A nutritional formulation including, per serving size (e.g., a 40-50 gram scoop of powder in water, or like): 15-20 (e.g., 18) g protein, 1-5 (e.g., 2) g fat, 5-15 (e.g., 11) g carbohydrates, as well as vitamins and minerals. In some embodiments, the nutritional formulation is replaced with a prebiotic formulation.

Although several ingredients are specifically recited herein, substitute ingredients in alternative embodiments are also contemplated. For example, in one embodiment, the cleansing formulation comprises one or more of rhubarb root, senna, buckthorn, magnesium, and aloe as a natural laxative that can be used instead of or in addition to cascara sagrada.

In some embodiments, one or more antibacterial herbs can be used instead of or in addition to yellow dock, licorice root, oregano leaf, and Oregon grape. These include but are not limited to Barberry (Berberis vulgaris)/Indian Barberry (Berberis aristatal), Berberine HCI, Calendula (Calendula officinalis), Chamomile (Matricaria recutita), Cinnamon (Cinnamomum verum), Clove (Syzygium aromaticum), Common Agrimony (Agrimonia eupatoria), Fennel (Foeniculum vulgare), Garlic (Allium sativum), Goldenrod (Solidago virgaurea), Goldenseal (Hydrastis canadensis)/Yellow Root (Xanthorhiza simplicissima), Meadow Sweet (Filipendula ulmaria), Sage (Salvia officinalis), Stinging nettle extract (Urtica dioica), and Turmeric (Curcuma longa).

In some embodiments, one or more other detoxification ingredients can be used instead or in addition to milk thistle for detoxification. These include but are not limited to Artichoke (Cynara scolymus), Parsley (Petroselinum crispum), Triphala (Terminalia chebula, Terminalia belerica, Emblica officinalis), and Uva Ursi (Arctostaphylos uva-ursi)

Fenugreek (Trigonella foenum-graecum) and/or Slippery elm (Ulmus rubra) may be used in some embodiments as ingredients for digestive support in addition or instead of the digestive ingredients identified herein,

The following one or more ingredients may be used in addition to, or instead of, ginger and peppermint for carminative support: Chamomile (Matricaria recutita), Cinnamon (Cinnamomum verum), Clove (Syzygium aromaticum), Fennel (Foeniculum vulgare), Goldenseal (Hydrastis canadensis)/Yellow Root (Xanthorhiza simplicissima), Licorice (Glycyrrhiza glabra), Oregon Grape Root (Mahonia aquifolium), Sage (Salvia officinalis), Artichoke (Cynara scolymus), Burdock (Arctium lappa), Dandelion (Taraxacum officinale), Parsley (Petroselinum crispum), Fenugreek (Trigonella foenum-graecum), Marshmallow (Althaea officinalis), and Slippery elm (Ulmus rubra).

As another example, in one embodiment, the prebiotic formulation may include other insoluble and soluble fibers (e.g., water-soluble polysaccharides and/or hemicellulose compounds). In another embodiment, inulin may be derived from a source other than Jerusalem artichoke. Glucomannan may be derived from a source other than konjac root (e.g., salep). In one embodiment, the prebiotic formulation includes 200-1000 mg of glucomannan and 200-1000 mg of inulin, among other optional ingredients. In one embodiment, the prebiotic formulation includes 200-1000 mg of soluble fiber and 200-1000 mg of insoluble fiber, with ranges in between provided for each ingredient in some embodiments, In some embodiments, acacia fiber/gum, Larch Arabinogalactan, oligofructose, Galactooligosaccharides, pectin, guar gum, and combinations thereof may be used in addition to or instead of glucomannan and inulin. In several embodiments, resistant maltodextrin (such as Fibersol-2® or alternate) is included in addition to or as a substitute for several or all of the ingredients in the prebiotic formulation. In one embodiment, the prebiotic consists or consists essentially of resistant maltodextrin.

As another example, in one embodiment, the probiotic formulation may include other bacterial strains. For example, in one embodiment, different strains of Bifidobacteria and Lactobacillus are used instead or in addition to the strains identified herein. Other beneficial strains that, in alternative embodiments, can be used are used instead or in addition to the strains identified herein include Lactobacillus acidophilus, Lactobacillus salivarius, Bifidobacterium bifidum, Bifidobacterium longum, Bifidobacterium breve, Lactobacillus casei and Lactobacillus fermentum. Suitable amounts with respect to the other bacterial strains are provided in arrange between 10-50%. In some embodiments, L. acidophilus, L. salivarius, L. casei, B. breve, L. paracasei, S. thermophilus, Lactococcus lactis, B. longum, L. bulgaricus, B. bifidum, and combinations thereof may be used in addition to or instead of the strains identified herein.

In yet another example, in one embodiment, the nutritional formulation may include protein sources other than whey (instead of or in addition to whey). Soy, egg, pea, hemp, and/or rice protein may be substitutes in one embodiment. Gelatin may be also used. A combination of individual amino acids may be provided in certain embodiments.

In several embodiments, the invention can be described in general terms. For example, in one embodiment, the invention generally comprises a system for changing the intestinal micro-organism environment through the use of a laxative to reduce the overall micro-organism load, a prebiotic fiber as a supportive substrate for the desired bacteria, and a probiotic to provide the desired bacteria. A nutritional formulation to support the beneficial environment may also be used. This system, in turn, fundamentally changes the intestinal flora from a microbiome that contributes to weight gain to a microbiome that contributes to weight loss/maintenance of a healthy weight.

In those embodiments intended to affect weight, many embodiments are directed to facilitating weight loss. However, it is contemplated that the microbiome is rebalanced to achieve a healthy state—including, for those individuals that have an unhealthy low BMI, an increase in weight (e.g., through building of lean muscle mass). In other words, for individuals who have an unhealthy microbiome that contribute to malabsorption of nutrients, the formulations described herein can be useful as well.

Although four formulations are contemplated according to certain embodiments, alternative embodiments comprise one, two or three of the individual formulations. In one embodiment, the system includes a cleansing formulation, a prebiotic formulation, and a probiotic formulation. In addition, although the formulations are referred to herein as first, second, third and fourth, it is contemplated that the order may be altered or that formulation may be taken sequentially or simultaneously.

In some embodiments, the system of the cleansing, prebiotic and probiotic formulations, and optionally the nutritional formulation, reduces undesired microorganism in the GI tract. In one embodiment, the ratio of undesired bacteria to desired bacteria changes from about 5:1 -10:1 to 1:5 -1:10. In one embodiment, the percentage of undesired bacteria (as compared to desired bacteria) is 40-95% pre-treatment and 1-20% post-treatment. In one embodiment, Helicobacter pylori is reduced. In some embodiments, the ratio of Firmicutes and Bacteroidetes are beneficially altered post-treatment. In one embodiment, excessive populations of undesired strains increase the storage of fat in adipose cells, and this fat storage is reduced (via the 10-80% reduction of such bacteria) post-treatment of the cleansing, prebiotic and probiotic formulations, and optionally the nutritional formulation, described herein. Undesired members of bacteria include the Streptococcus and Clostridium families. In some embodiments, the system of the cleansing, prebiotic and probiotic formulations, and optionally the nutritional formulation, reduces undesired bacteria (e.g., Streptococcus and/or Clostridium strains) by 10-20%, 20-30%, 30-50%, 50-75%, 30-90%, and overlapping ranges thereof.

In several embodiments, the overall diversity in the microbiome is increased through use of the formulations described herein. In some embodiments, the diversity (or the number of different types of bacteria) in the GI tract is increased by 10-25%, 25-50%, 50-75%, 75-100%, 3-10 fold, and overlapping ranges thereof.

In some embodiments, the system of the cleansing, prebiotic and probiotic formulations, and optionally the nutritional formulation, alters the percentage of gram-negative bacteria and/or the percentage gram-positive bacteria by 10-20%, 20-30%, 30-50%, 50-75%, 30-90% and overlapping ranges thereof.

According to several embodiments, undesired microorganisms other than bacteria are also reduced through the use of one or more of the formulations described herein. For example, the system of the cleansing, prebiotic and probiotic formulations, and optionally the nutritional formulation, may reduce Candida albicans by 10-90%.

The formulations described herein can provide both short-term and long-term benefits according to several embodiments. Beneficial effects from use of the formulations described herein occur upon use, within hours or days of use, or within about a few weeks of use. In several embodiments, the health of the GI tract continues to improve over about 2-4 weeks, about 4-6 weeks, about 6-10 weeks, or about 12-16 weeks (or longer) of use, A 30-day, 60-day or 90-day weight management program that includes all four formulations are provided in several embodiments. In one embodiment, a kit comprising a cleansing formulation, a prebiotic formulation and a probiotic formulation is provided, along with directions for use. In one embodiment, the kit additionally comprises a nutritional formulation. In some embodiments, the formulations are sold separately but instructions for use are provided to use the individual formulations as part of a three or four step regime.

Directions for using the formulations described herein can be included with the formulations. Such directions include, but are not limited to, the following: With respect to the cleansing formulation, one serving (e.g., 2 capsules) is taken with at least 8 oz. of liquid (e.g., water) in the morning and 2 capsules with 8 oz. of liquid (e.g., water) in the evening for 7 to 21 (e.g., 14) days. With respect to the prebiotic formulation, one serving (e.g., 2 capsules) is taken with at least 8 oz. of a liquid (e.g., water) 30 to 45 minutes before meals, three times a day. With respect to the probiotic formulation, one serving (e.g., 2 caplets or 1-2 spheres) are taken 1-2 times a day. With respect to the nutritional formulation, 1 serving size (e.g., 1 scoop of about 30-60 grams) is mixed with at least 8 oz. of a liquid (e.g., water, low-fat milk, almond milk or soy milk, etc.) and ingested 1-4 times a day. In one embodiment, it is instructed that the cleanse formulation and the probiotic formulation not be taken simultaneously.

According to several embodiments, the formulations described herein are designed for oral administration. Tablets, capsules, caplets, spheres, powders, gels, and liquids are provided in some embodiments. Some of the formulations (e.g., the vitamin portion of the nutritional formulation) may be provided via injection. Other modes of administration (e.g., injectable, oral, topical, suppository, etc.) are also contemplated.

In some embodiments, the invention comprises one of the cleansing, prebiotic, probiotic and nutritional formulations, as described herein. In other embodiments, two of the cleansing, prebiotic, probiotic and nutritional formulations, as described herein, are provided.

Terms used herein, including the claims, are generally intended as “open” terms (for example, the term “including” should be interpreted as “including but not limited to,” the term “having” should be interpreted as “having at least,” the term “includes” should be interpreted as “includes but is not limited to,” etc.). The use of articles such as “a” or “an” should not be unduly limited to a singular element. In addition, even if a specific number of an introduced claim recitation is explicitly recited, those skilled in the art will recognize that such recitation should be interpreted to mean at least the recited number (for example, the recitation of “two compounds,” without other modifiers, means at least two compounds or two or more compounds). Further, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). It will be further understood by those within the art that any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” The phrase “consistently essentially of” X, Y and Z may read on embodiments that contain compounds other than X, Y or Z that do not materially affect the basic and novel characteristics of the formulation or claim (such as biologically inactive ingredients, including excipients, colorings, flavors, inert additives, etc.). In several embodiments, the formulations described herein consist or consist essentially of the active and/or functional ingredients identified herein (e.g., ingredients that provide at least one of the following functions: cleansing, digestive support, detoxification, carminative, prebiotic, probiotic, etc.).

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. As will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth. The phrases “and ranges in between” or “overlapping ranges thereof” can include ranges that fall in between the numerical values listed. For example, “1, 2, 3, 10, and ranges in between” can include 1-10, 1-3, 2-10, etc. and “1-5, 5-10, 10-15” can include 1-10, 5-15, etc.)

What is claimed is:

1. A system of formulations for oral administration for beneficially altering the microbiome, comprising:
(i) a cleansing formulation;
(ii) a probiotic formulation; and
(iii) a nutritional formulation;
wherein the cleansing formulation comprises cascara sagrada, Oregon grape, milk thistle extract, oregano leaf, peppermint leaf, licorice root, ginger, marshmallow, burdock, dandelion, and yellow dock,
wherein the probiotic formulation comprises inulin, Lactobacillus plantarum, Lactobacillus rhamnosus, Lactobacillus gasseri, Bifidobacterium infantis, Bifidobacterium lactis, and Bifidobacterium breve, and
wherein the nutritional formulation comprises whey protein, resistant maltodextrin, erythritol, a plant-based oil, vitamins and minerals.

2. The system of claim 1,
wherein the ingredients in said cleansing formulation are provided in the following amounts per serving size:
wherein said cascara sagrada is provided in a range of 100-400 mg,
wherein said Oregon grape is provided in a range of 200-400 mg,
wherein said milk thistle extract is provided in a range of 50-150 mg,
wherein said oregano leaf is provided in a range of 50-150 mg,
wherein said peppermint leaf is provided in a range of 20-100 mg,
wherein said licorice root is provided in a range of 20-100 mg,
wherein said ginger is provided in a range of 5-75 mg,
wherein said marshmallow is provided in a range of 50-200 mg,
wherein said burdock is provided in a range of 50-150 mg,
wherein said dandelion is provided in a range of 50-150 mg and
wherein said yellow dock is provided in a range of 50-150 mg.

3. The system of claim 2,
wherein the probiotic formulation comprises the following ranges provided in colony forming units per serving size:
said Lactobacillus plantarum in a range of 2-20 billion,
said Lactobacillus rhamnosus in a range of 2-15 billion,
said Lactobacillus gasseri in a range of 0.5-10 billion,
said Bifidobacterium infantis in a range of 0.5-5 billion,
said Bifidobacterium breve in a range of 0.5-5 billion; and
said Bifidobacterium lactis in a range of 0.5-5 billion.

4. The system of claim 1,
wherein the cascara sagrada is provided in a range of 100-400 mg, and
wherein the nutritional formulation comprises between 10-30 grams of aid whey protein.

5. The system of claim 1, further comprising a prebiotic formulation.

6. The system of claim 5, wherein the prebiotic formulation comprises glucomannan, rice bran powder, barley grass powder, and Jerusalem artichoke.

7. The system of claim 5, wherein the prebiotic formulation comprises resistant maltodextrin.

8. A system of formulations for oral administration for beneficially altering the microbiome, comprising:
(i) a cleansing formulation;
(ii) a probiotic formulation; and
(iii) a nutritional formulation;
wherein the cleansing formulation comprises cascara sagrada,
wherein the probiotic formulation comprises Lactobacillus and Bifidobacterium strains, and
wherein the nutritional formulation comprises whey protein and resistant maltodextrin,

9. The system of claim 8, wherein the cleansing formulation further comprises:
oregon grape in a range of 200-400 mg,
milk thistle extract in a range of 50-150 mg,
oregano leaf in a range of 50-150 mg,
peppermint leaf in a range of 20-100 mg,
licorice root in a range of 20-100 mg,
ginger in a range of 5-75 mg,
marshmallow in a range of 50-200 mg,
burdock in a range of 50-150 mg,
dandelion in a range of 50-150 mg, and
yellow dock in a range of 50-150 mg,
wherein said amounts are provided per serving size.

10. The system of claim 8, further comprising a prebiotic formulation, wherein the prebiotic formulation comprises:
glucomannan,
rice bran powder,
barley grass powder, and
Jerusalem artichoke.

11. The system of claim 8, further comprising a prebiotic formulation, wherein the prebiotic formulation comprises a resistant maltodextrin.

12. The system of claim 8, wherein the Lactobacillus and Bifidobacterium strains of the probiotic formulation comprise:
Lactobacillus plantarum,
Lactobacillus rhamnosus,
Lactobacillus gasseri,
Bifidobacterium infantis
Bifidobacterium lactis, and
Bifidobacterium breve.

13. The system of claim 9, wherein the Lactobacillus and Bifidobacterium strains of the probiotic formulation comprise at least one of the group consisting of: Lactobacillus plantarum, Lactobacillus rhamnosus, and Lactobacillus gasseri and at least one of the group consisting of: Bifidobacterium infantis, Bifidobacterium lactis, and Bifidobacterium breve.

14. The system of claim 8,
wherein the Lactobacillus and Bifidobacterium strains of the probiotic formulation comprise, per serving size:
Lactobacillus plantarum in a range of 2-20 billion colony forming units,
Lactobacillus rhamnosusin a range of 2-15 billion colony forming units,
Lactobacillus gasseri in a range of 0.5-10 billion colony forming units,
Bifidobacterium infantis in a range of 0.5-5 billion colony forming units,
Bifidobacterium breve, in a range of 0.5-5 billion colony forming units, and
Bifidobacterium lactis in a range of 0.5-5 billion colony forming units.
